(19) United States
(12) Patent Application Publication
(10) Pub. No.: US 2024/0350757 A1
(43) Pub. Date: Oct. 24, 2024

(54) TIDAL VOLUME, PRESSURE, INSPIRATORY TIME, AND VENTILATION RATE MEASUREMENT DEVICE DURING MANUAL VENTILATION

(71) Applicant: safeBVM Corp, Boston, MA (US)

(72) Inventor: Prathamesh PRABHUDESAI, Fremont, CA (US)

(73) Assignee: safeBVM Corp, Boston, MA (US)

(21) Appl. No.: 18/628,411

(22) Filed: Apr. 5, 2024

Related U.S. Application Data
(63) Continuation of application No. PCT/US22/77783, filed on Oct. 7, 2022.
(60) Provisional application No. 63/253,243, filed on Oct. 7, 2021.

(51) Int. Cl. A61M 16/00; A61M 16/20

(52) U.S. Cl. CPC A61M 16/024; A61M 16/0051; A61M 16/0084; A61M 16/208; A61M 2016/0027; A61M 2016/0033; A61M 2205/3368

(57) ABSTRACT

A “bag ventilator assembly” includes a compression bag, a manifold, a breathing interface, and a sensor. The compression bag is configured to be attached to deliver a breathing gas to the manifold and has an air inlet and an air outlet. The air inlet includes a one-way valve that allows air to flow into the compression bag from the ambient as the compression bag expands, and the manifold is connected to the air outlet of the compression bag. The breathing interface is configured to be attached to the air outlet of the manifold to receive the breathing gas from the manifold, and the sensor is configured to be secured to the air inlet on the compression bag such that the sensor can sense air entering the compression bag while remaining isolated from exhaled air.

### CROSS-REFERENCE TO RELATED APPLICATIONS

This application is a continuation of PCT Application No. PCT/US22/77783 (Attorney Docket No. 56075-704.601), filed Oct. 7, 2022, which claims the benefit of U.S. Provisional No. 63/253,248. (Attorney Docket No. 56075-704.101), filed Oct. 7, 2021, the entire content of each of which is incorporated herein by reference.

### BACKGROUND OF THE INVENTION

### 1. Field of the Invention

The present invention relates generally to resuscitators and manual ventilation devices used for artificially ventilating patients who are unable to breathe adequately on their own. More particularly, the present invention relates to the placement of sensors on such resuscitators and manual ventilation devices.

Typical manual ventilation devices comprise an inlet which allows air to fill a bag or air chamber. In many cases ambient air (at 21% Oxygen concentration) is adequate to maintain ventilation and oxygenation of the patient. However, in some situations a higher concentration of oxygen is required. In these cases, a reservoir and source of concentrated oxygen may be utilized. The inlet conventionally includes a valve system to ensure unidirectional flow of air to the patient. The bag chamber is typically built to ensure that it can be compressed easily to push air contained in the chamber through the outlet only in the direction of arrows displayed above. The bag chamber often serves as a temporary gas repository until the gas is pushed out to the patient via the outlet.

A standard “bag-ventilator-mask” (BVM) 50, as illustrated in FIG. 1, includes a compression bag 52, a reservoir bag 54 connected to the compression bag by a reservoir valve 56, a positive end expiratory pressure (PEEP) valve 58, and a pop-off valve 60. The compression bag 52. PEEP valve 58 and pop-off valve 60 are all connected to a manifold 62 having a connector port 64 for removably receiving a breathing mask 70. A breathing mask 70 typically has an air cushion 72 on one surface for placing over a patient's mouth and nose when in use and a connector fitting 74 for detachably mating to the connector port 64 of the manifold 62. Commonly, the connector port 64 on the manifold 62 will be a male connector and the connector fitting 74 on the breathing mask 70 will be a female fitting. The connector port 64 will often also include an inner female connector to allow for connection to an endotracheal tube m place of the breathing mask, and the present invention can be adapted to work with type of patient interface and any pair of detachable connectors, including screw connectors, bayonet connectors, latched connectors, and the like.

During use of BVM such as that shown in FIG. 1 typically comprises squeezing the bag/air chamber where the volume delivered depends on the extent of the squeeze. There is slight compression of the air until the output resistance is overcome but this decrease in volume of the bag (dependent on the size and type of the squeeze) produces a (roughly) equal volume of air to be pushed through the outlet. When the provider releases the bag, the elastic walls of the bag bounce back to their resting state (in self-inflating bags) generating a negative pressure inside the bag During this phase, this negative pressure results in refilling of the bag via the inlet valve (also a one-way valve).

Proper control of tidal volume, pressure, inspiratory time, and ventilation rate during manual ventilation is critical to avoid patient injury and to ensure proper ventilation. There is a clear benefit from the availability of a means of objective determination of ventilation patterns.

Mechanical ventilators that function autonomously by default can measure these parameters Being automated, they are able to finely control artificial ventilation. A healthcare provider controlling manual ventilation generally doesn't have access to quantitative feedback and may use signs like chest rise to evaluate if they are delivering the optimal tidal volume. This is very subjective, and often results in under-ventilation or over-ventilation. Overventilation can result in “volutrauma” due to high tidal volumes, gastric insufflation and barotrauma due to higher pressures. Gastric insufflation results in vomiting, aspiration, aspiration pneumonia and ARDS.

Such one-size-fits-all approaches, while better than nothing, are inadequate to accommodate the range of physiological variation and disease states that each patient may present. For example, obstructive and restrictive diseases will create the need for higher pressures for adequate ventilation Pop-off valves and manometric readings may not be well representative of this need for higher pressure, which may then result in underventilation.

Hyperventilation decreases CO₂ in the body of a given patient, which results in alkalosis. Alkalosis impedes the patient's blood hemoglobin to bind to oxygen, which ultimately gives rise to potentially fatal conditions such as cerebral hypoxia and hyperventilation syndrome, which may lead to brain injury and patient mortality. Alkalosis also causes vasoconstriction, which may lead to decreased blood flow to the brain and has been shown to result in worse outcomes in patients with traumatic brain injuries. Furthermore, inappropriate tidal volumes or the total volume of air that is given with each breath, may lead to barotrauma and the development of Acute Respiratory Distress Syndrome (ARDS), which leads to increased morbidity and mortality. Recommended airflow parameters for ARDS patients according to the ARDSnet protocol include low tidal volumes; starting at 6 ml/kg PBW and plateau pressure≤30 cmH2O.

An estimated 13.1 million BVMs are used annually in the U.S. Despite being the gold standard of emergency airway management and artificial respiration, these resuscitators frequently are used improperly, even by experienced providers, potentially leading to poor patient outcomes. One study found that providers delivered unsafe manual ventilation 81% of the time.² Overventilation can arise from high tidal volume and pressure delivery and result in air being pushed into the stomach as the pressure crosses the lower esophageal sphincter (LES)'s opening pressure of 18-20 cm H₂O. As inspiratory pressure crosses 22 cmH₂O, gastric insufflation occurs. Onset of lung injury occurs in animal models at 30 cmH₂O. Barotrauma, interstitial edema, and inflammation occurs at 40 cmH₂O, pneumomediastinum at 57 cmH₂O, and pneumothorax at 62 cmH₂O. Other potentially fatal complications from improper ventilation technique include cyclic worsening of lung compliance and cardiopulmonary compromise arising from decreased cardiac filling due to high intrathoracic pressure. Gastric insufflation occurs up to 71% of the time while using a BVM, which leads to a 65% decrease in tissue oxygen delivery and commonly leads to vomiting and aspiration. One study showed that 33% of all treated out-of-hospital cardiac arrest patients exhibit vomiting, and the survival rate for out-of-hospital cardiac arrest is only 10%. Patients who did not respond to cardiopulmonary resuscitation (CPR) had an incidence of pulmonary aspiration of 29% when assessed at autopsy. Patients who survive go on to develop aspiration pneumonia 50% of the time. The mortality rate from aspiration pneumonia is largely dependent on the volume and content of aspirate but mortality can reach up to 70%. Approximately 33% of patients with aspiration pneumonitis develop a more severe, protracted course associated with acute lung injury (ALI) and acute respiratory distress syndrome (ARDS), which has a mortality rate of 43%. Moreover, the cost of care for aspiration pneumonia and ARDS can range from $36,000 to $140,000 per patient A standard disposable resuscitation bag in the USA is priced at $12-$35. A high-fidelity sensor that can accurately measure flow, pressure, volume, and temperature thereby being able to calculate the other airflow parameters is priced at $95 or more. WO2011127123 discusses use of wireless sensors that are again placed between the connector and the outlet. This minimizes cost due to wiring to $70, however makes the sensor more complicated while increasing risk to the patient due to connectivity issues.

There is, therefore, a need for a simple and inexpensive device and method for monitoring respiration/ventilation of a person to overcome the above-mentioned problems associated with the prior art.

### Background Art

WO2014158726 and WO2016123562 illustrate sensors incorporated between the connector and the outlet. U.S. Pat. No. 6,203,502 describes a respiratory function monitoring device comprising a flow sensor and a conversion device. The device comprises two pressure transducers, one for measuring differential pressure corresponding to a gas flow rate, and a second to measure static airway pressure WO 95/06234 describes a differential pressure sensor for measuring respiratory gas flow. The sensor is designed to have the capability of accommodating a wide variety of gas flow inlet conditions while employing a minimum of added system volume or resistance to flow. U.S. Pat. No. 6,544,192 describes a patient monitoring apparatus for quantitatively measuring a physiological characteristic of a patient A first patient interface communicates with an airway of a patient such that substantially all gas inhaled and exhaled by the patient passes through the patient interface. One or more vent elements associated with the first patient interface communicate the first patient interface with an ambient atmosphere so that a pressure differential is created between the pressure in the first patient interface and the pressure of the ambient atmosphere. A sensor communicates with the first patient interface and measures a fluid characteristic resulting from this pressure differential and outputs a first signal indicative of that characteristic.

### SUMMARY OF THE INVENTION

The present invention provides sensors at the air inlet of a BVM or other manual resuscitator rather than at the outlet as found in the prior art. As used herein and in the claims, the acronym “BVM” is not limited to “bag-mask-ventilators” and is meant to refer to any manual bag ventilator, including those with breathing masks, endotracheal tubes, or other patient interfaces A primary benefit of incorporating sensors at the inlet (in contrast to the outlet) is that a sensor at the inlet can be reusable in contrast to a sensor at the outlet which typically must be disposable due to the regulatory guidelines and sterilization protocols for minimizing infections Locating sensors at the inlet also minimizes dead space and reduces accumulation of exhaled air in the air chamber which can adversely affecting oxygenation of the patient. Additionally, removing sensors from the flow path reduces the length of the circuit, thus reducing torque on the ET tube or airway that could potentially result in mispositioning of the airway.

While embodiments of the present invention are illustrated based upon bag ventilators intended for resuscitation using patent mask interfaces, the present invention will also find use with any device covered by ISO 5356 standards, including a variety of specific devices and components that may be used between the manual compression bag and the mask or other patient interface, not limited to resuscitators or mask interfaces.

In a first aspect, the present invention provides a “bag ventilator assembly” (BMV) comprising a compression bag, a manifold, a breathing interface, and a sensor. The compression bag is configured to be attached to deliver a breathing gas to the manifold and has an air inlet and an air outlet. The air inlet includes a one-way valve that allows air to flow into the compression bag from the ambient as the compression bag expands, and the manifold is connected to the air outlet of the compression bag. The breathing interface is configured to be attached to the air outlet of the manifold to receive the breathing gas from the manifold, and the sensor is configured to be secured to the air inlet on the compression bag such that the sensor can sense air entering the compression bag while remaining isolated from exhaled air.

In specific aspects of the present invention, the sensor may be configured to measure any one of pressure, volumetric flow rate, mass flow rate, and temperature.

In specific aspects of the present invention, the sensor may be configured to be sterilized by any one of heat, chemical, or gas sterilization.

In specific aspects of the present invention, the sensor may be configured to be detachably secured to the air inlet on the compression bag.

In specific aspects of the present invention, the sensor may be configured to be non-detachably secured to the air inlet on the compression bag.

In specific aspects of the present invention, the sensor may be configured to store or transmit information with a cable or wirelessly.

In specific aspects of the present invention, the sensor may be configured to alert the user or provide feedback based on the readings.

### BRIEF DESCRIPTION OF THE DRAWINGS

FIG. 1 illustrates a prior art resuscitation bag.

FIGS. 2A-1 to 2A-5, 2B, 2C, and 2D illustrate a first resuscitation bag constructed in accordance with the principles of the present invention.

FIG. 3 illustrates a second resuscitation bag constructed in accordance with the principles of the present invention.

### DETAILED DESCRIPTION OF THE INVENTION

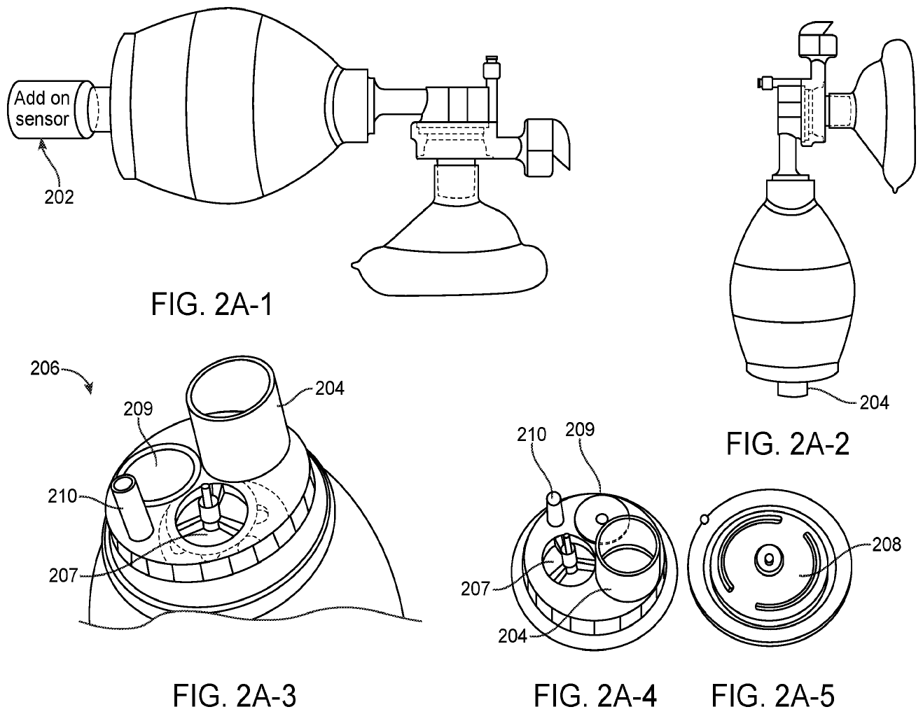

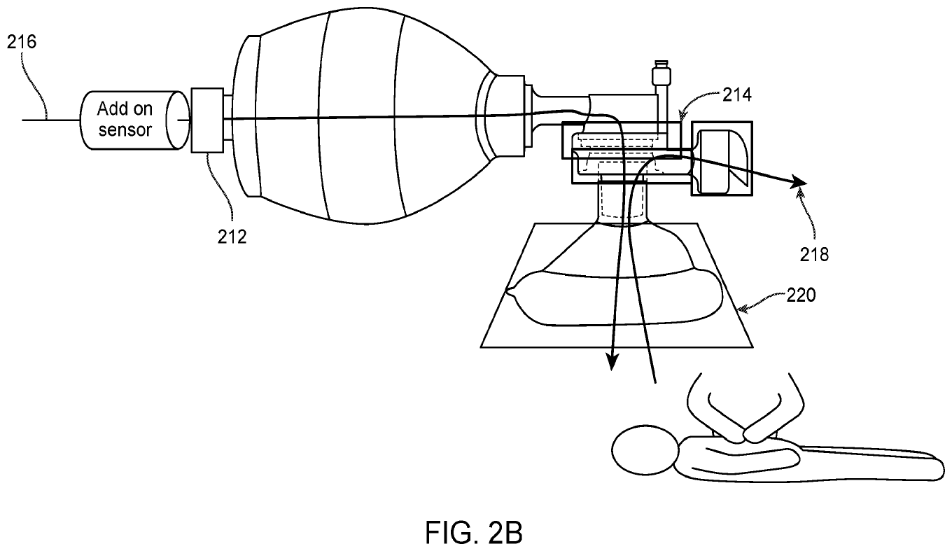

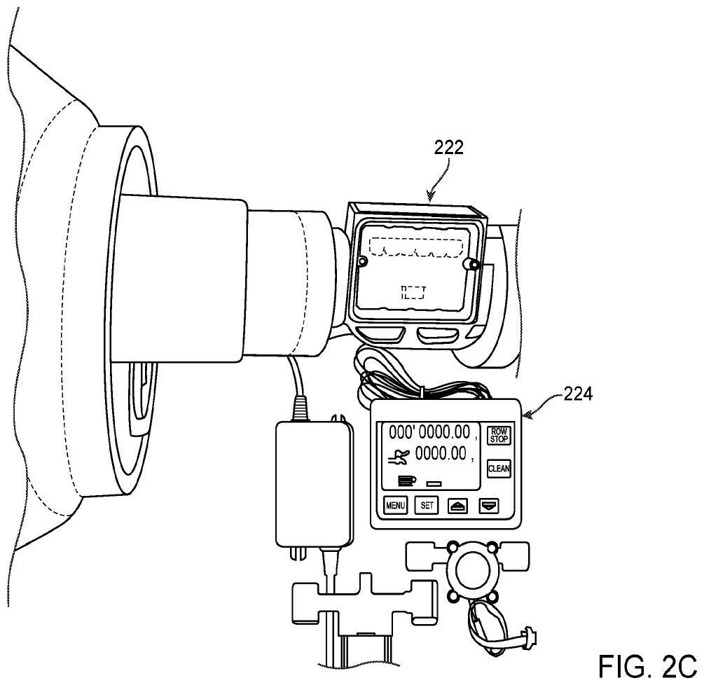

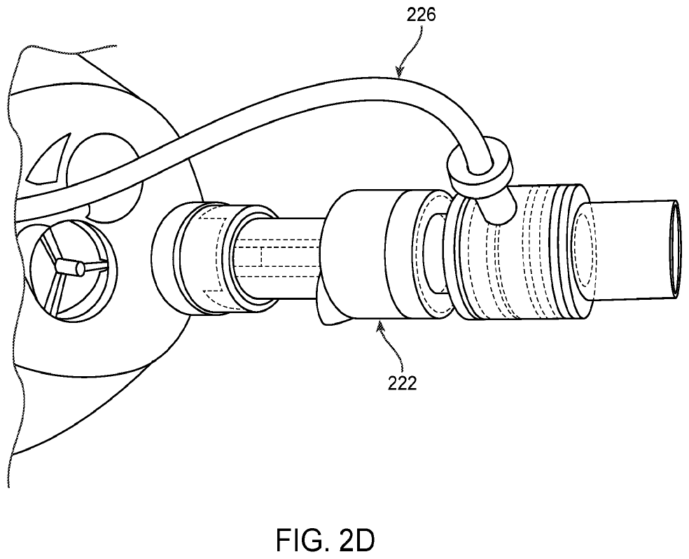

Resuscitation bags in accordance with the present invention provide sensors at the inlet that can be reusable. This enables the use of high-quality sensors that can effectively provide accurate information on important airflow parameters. Two possible embodiments of this idea are: (1) Addition of an adjunct flow sensor to an existing commercially available bag as shown in FIG. 2A and (2) Creation of a bag with a modified inlet to which reusable sensors can be attached as shown in FIG. 2B.

Particular embodiments of the present invention will incorporate general principle of using a measurement component on the air inlet to the bag in contrast to on the air outlet as taught by the prior art. The “air inlet,” can be any port, passage, valve, fitting, or the like configured to receive the inlet air from the ambient as the bag expands. The air inlet will be located outside of all passageways and other components which are exposed by the patient exhalation.

Currently, the technology for measuring airflow parameters uses sensors in the flow path that is in communication with a patient's lungs. These sensors are positioned between the airway/mask and the bag. The sensor assembly may or may not have a display. If the display is not present on the sensor, an adjunct monitor's display is typically utilized. US20180147375 which is currently abandoned utilizes a detachable sensor module.

Many compact flow sensors are typically based on the Venturi principle, i.e., the pressure drop associated with a flow restriction is measured. However, in order to achieve a high level of accuracy, this method requires a restriction with a very sophisticated geometry. This may often be forbiddingly expensive for a single-use unit. Single-use is normally desired or required for respiration measurements due to the risk of cross-contamination between patients.

The Venturi principle also has the disadvantage that two pressure outlets are needed from the restriction, which may complicate the geometry and increase cost. In addition, two pressure sensors are needed to measure the pressure drop in the restriction, which may increase cost. Alternatively, the two pressure sensors may be replaced with a single differential pressure sensor. If a differential pressure sensor is used, the output from the sensor can, however, only be used to assess flow, and not to monitor the absolute airway pressure, which may be important for detecting mask leakage, airway occlusion etc. Additionally, Venturi measurements are also known to be unstable in conditions of turbulent flow. VentCheck™ by Respironics is a hand-held respiratory mechanics monitor that measures flow and pressure at the patient's airway. This monitor uses differential pressure single-use flow sensors to provide a breath-by-breath picture of the patient's respiratory status.

The present invention is based on the following principles:

The most accurate way to document key airflow parameters like tidal volume and pressure in the alveoli is by directly quantifying flow, pressure, and temperature by using in-situ implantable sensors. At this point though it is technologically feasible however cost prohibitive. Also, currently there are no devices on the market that are commercially available for this intended use.

Another very accurate way to quantify tidal volume and rate is by documenting change in volume of chest (also known as chest expansion) and how many times that happens in a minute. As previously discussed, tidal volume can be measured using Electrical impedance tomography as discussed by Frerichs et al in their paper “Electrical impedance tomography a method for monitoring regional lung aeration and tidal volume distribution?”. However, these methods also have drawbacks because they interfere with chest compression.

So instead of implanting a sensor, the sensor can be placed just outside/proximal to the patient's face. However, this sensor has to be disposable because it is in contact with the patient's humidified gas pathway. A proximal flow sensor that is temperature controlled can easily be utilized to monitor pressure in the air channel too. Some of the inventions described above do that. Sensirion SFM 3300-D with a pressure evaluation circuit is a commercially available disposable OEM device that can be integrated into monitors or ventilators. The consumable cost on this product when available with large distributors is $38.5/unit for the flow sensor, $95/cable, $16.5/unit for pressure kit thus bringing the total to $150. Even at scale for a business manufacturing all these components in-house, the assembly would still cost $70+. This is almost 4 times the price of an average bag valve mask. Additionally, when these sensors are used with a mask, their efficacy is dependent on factors like mask seals not causing a leak.

Another way to determine the tidal volume delivered to the patient is by measuring the exhaled tidal volume at the exit port. This exit port located at (58) can be utilized for measuring volume. This measurement is also affected by mask leak.

Incorporation of sensor of mechanical structure within or on the bag. This apparatus could determine the change in volume of the bag. Change in volume of the bag will be almost exactly equal to the volume or gas displaced and delivered through the outlet. However, calibrating sensors and ensuring accuracy in an elastic collapsible chamber is challenging. Also, any sensor on the exterior of the bag will find it challenging to measure pressure in communication with the patient's lungs.

A principal object of the present invention is to provide a method for obtaining key airflow parameters through a sensor placed at the inlet to the bag. The actual sensors could be implemented via a variety of mechanical, electronic or hybrid technologies. Operative principles:

The bag has a fixed volume.

The bag has an inlet with a mechanism such that air never flows out of the inlet when the bag is squeezed.

In the above situation, when a user squeezes the bag, the volume of the bag will reduce:
a. Change in volume of bag (compression)=Air pushed out of the outlet;
b. Change in volume of bag (re-expansion)=Air drawn inside the bag chamber;

As long as the user is performing ventilation according to clinically accepted standards,
a. Change in volume of bag (compression)=Change in volume of bag (re-expansion)

Thus, by utilizing a measurement mechanism for air flow at the inlet, we will be able to document several airflow parameters. Furthermore, because the inlet is in mechanical communication with the bag, we will be able to be in fluid communication with the bag chamber simply by bypassing the unidirectional valve present at the inlet.

This also opens up the opportunity to quantify pressures in the bag chamber. There is anecdotal evidence for very experienced Respiratory Technicians (RT) being able to determine the compliance and resistance of the lung based on the feel of the bag. Sensors measuring pressure in the bag chamber will make the feel of the bag more objective, potentially enabling us to determine disease states of the lung based on different airflow parameters.

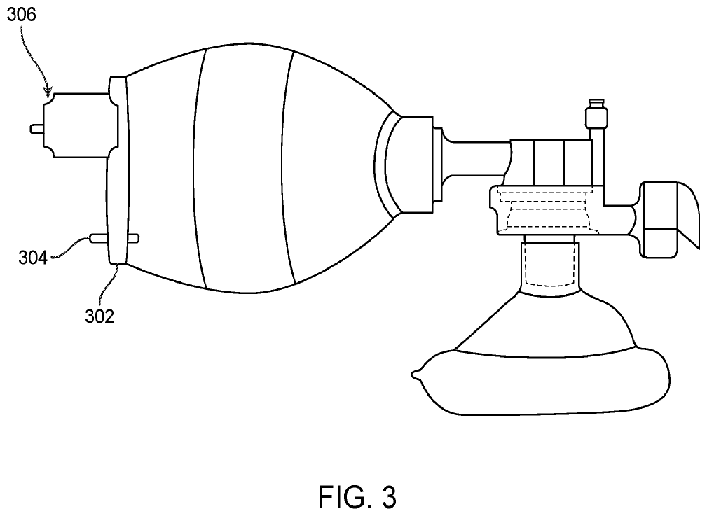

In case the invention is electronic it may incorporate one or more of the following: the flow sensor can include a thermal mass flow sensor element, the chip may contain an amplifier. A/D converter. EEPROM memory, digital signal processing circuitry, and interface. The invention may also include a pressure sensor. Image B in FIG. 3 goes over modification of the entire inlet assembly for building a comprehensive volume and pressure evaluation system.

A filter or other sterile barrier may be used to ensure additional protection from contamination for the reusable sensor.

A completely disposable assembly utilizing differential pressure calculations at the inlet is also possible. For the purely mechanical use case, a pressure syringe and timer could be incorporated. A variety of mechanical pneumatic control devices can be applied to regulate the inlet air and perform various calculations on it.

There is also an opportunity to utilize a combination of disposable and reusable parts to obtain higher accuracy while reducing costs. In this hybrid assembly, we can also add a timer or a flashlight that lets the user know the ventilation rate or instructs the user to deliver air to match a predetermined rate. A disposable/reusable assembly provides the opportunity to add a display to the device. It may have a processor that is configured to receive the measurement from the sensor and provide information on the display based on the received measurement. The information recorded and displayed may include a current breath rate, a pressure-vs-time curve, and guidance to the user to assist in achieving a target breath rate. The device could also have logging capacity with a timestamp. This display can help users improve their technique to better achieve target treatment parameters. Such sensors can further be incorporated into systems and apparatus to measure, store, and transmit, ventilation data and provide feedback to the provider on technique.

In the case of an electronic device the inlet comprising of a sensor, there is further opportunity to quantify mask leak by incorporating a similar sensor at the exhalation port.

Mask leak=Volume of air refill detected at the inlet−volume detected at exhalation port.

### Ability to Function Closer to a Ventilation

A mechanical ventilator in its most basic form has a volume control mode. These ventilators are frequently used in transporting patients and have very basic functionality. The clinician can set a tidal volume and a rate. The ventilator delivers that tidal volume at the set rate (12 breath/min) at a fixed flow rate. More complex transport ventilators have a decelerating pattern with a peak of around 40 L/min. These waveforms mimic human breathing. In the proposed invention, the provider will be able to observe the tidal volume of the breath by looking at the inflow volume into the bag. This volume may be visualized by looking at the screen. By detecting the number of breaths, the provider will be able to gauge the rate too. SafeBVM's Sotair device as described in WO2019232491 has control over pressure and flow. Thus, all the essential parameters of ventilation are being regulated enabling a human provider to deliver airflow with a ventilator precision.

In some embodiments, a pressure, flow rate, and/or time sensor may be disposed at the input side.

In some embodiments, the mechanical ventilators of the present invention may be disposable, may be non-disposable, may be mechanical, may be electronic, have filters, and the like.

Calculations may be made to determine the tidal volume. For example, tidal volume may be determined by a simple integration of the flow rate over time, e.g., time measurement multiplied by flow rate based on the differential pressure provided by the resiliency of the bag. Others might involve the measurement of flow rate, either based on differential pressure, hot wire anemometer, or other known flow measurement techniques, as well as other known ways of measuring the tunnel volume at the inlet.

Calculations for tidal volume as by flow rate can be made as follows:

Step 1: Identify and isolate breaths based on changes in flow patterns. Flow at our sensor will always be unidirectional. A flow pattern will be identified as the in-flow rate crosses +2 L/min, reaches a peak, and drops to +2 L/min, later returning to 0. This pattern will be considered 1 breath. A+2 L/min threshold helps minimize artifacts.

Step 2: Integrating flow rate at each time interval will provide volume. For e.g., if we are reading at a 10 ms interval, the calculation would be:

$$Volume ( ml ) = \frac{Sum of all flow rates readings in L / min at each 10 ms reading}{6}$$

Calculations for tidal volume as by pressure differential can be made as follows:

Step 1: Identify and isolate breaths based on changes in pressure patterns. Pressure at our sensor will always be at a resting value either when the manual resuscitator is inactive or is in the compression state. When the provider releases the compression, the process of manual resuscitator coming back to its resting state will initiate. During this stage, immediately following the release of the compression, there is a negative pressure within the bag. The properties of this negative pressure are related to the material properties/recoil properties of the bag and can be denoted by K. Each inflow breath can thus be identified and isolated by −20 Pa change in baseline (start of inflow) with a peak reaching up to −600 Pa and then return to baseline (end of inflow) as the bag is completely refilled with air from the surroundings.

Step 2: Because the refilling of the bag is completely passive and doesn't have an outside force affecting it, the volume entering the bag/tidal volume is a function of the duration of passive refilling (refill time) and recoil properties of the bag and may be calculated as:

Volume (ml)=K(recoil properties)×(Time at end of inflow−Time at start of inflow)

The above-mentioned time calculation could be either mechanical or electronic. In a mechanical version a pressure syringe can be utilized.

$$s = {V}_{i} t + 1 / 2 {at}^{2}$$

$$S ( Syringe with distance calibrated tidal volume ) = 0 + 1 / 2 \frac{Pressure \times Area of Piston}{Mass of piston} \times {t}^{2}$$

Acceleration at the piston may not be constant and will have to be integrated. Or the pressure value being so small, there is potential to ignore it for simplicity of design and similar functionality.

The basic measurement is flow rate versus time through inlet valve during refill. This will enable us to calculate the total flow by integrating the flow rate over time (either by electronic or mechanical means). In a situation where there are no leaks through the mask, this total flow directly gives you the delivery volume. Since the desired delivery volume can vary considerably based simply on size of the individual, a basic level of customization is immediately available to the provider.

If a timer type device is incorporated, either mechanical (kitchen timer) or electronic, there is an immediate possibility of prompting the responder to deliver at a desired respiratory frequency.

The measurement of pressure inside the bag (which would allow pressure monitoring during delivery also) opens up another range of possibilities such as detecting downstream obstructions or other anomalies. Some limited detection of disease states may also be possible though due to the limited number of measurement parameters it may be hard to disambiguate similar disease states. Some of the machine learning type approaches are broadly applicable here but the small number of variables makes it more like garden-variety classification rather than something more elaborate. These pressure in addition to the other parameters the sensor is measuring can be analyzed further to quantify and predict air leak or air entering the patient.

The pressure sensor can be placed behind a sterile barrier to avoid contamination (as it will be connected to the patient's airway during delivery).

The valve systems both at the inlet and outlet need to be a sterile barrier for the use of reusable sensors.

Flow may be measured at the inlet, typically indirectly based upon velocity (from which you back out volume using flow area) or mass (from which you can back out volume with reasonable assumptions around density). Useful flow sensors include: Vane Sensors; Deflecting Plate Sensors; Differential Pressure Sensors; Ultrasonic Flow Sensor; Coriolis Flow Sensors (Mass); Hot Wire Anemometers (Mass); Cold Wire Sensors (Mass); and Karman Vortex (Mass).

Pressure may be measured at the inlet using any one of a variety of pressure transducer sensing technologies, most commonly Electronic Pressure Transducers (usually based directly or indirectly on the piezoelectric principle); Fluid-filled devices (barometer/manometer/micromanometer), potentially combined with electronics for readout; Diaphragm Pressure Gages; Mechanical Pressure Gages such as a Bourdon tube; and the like

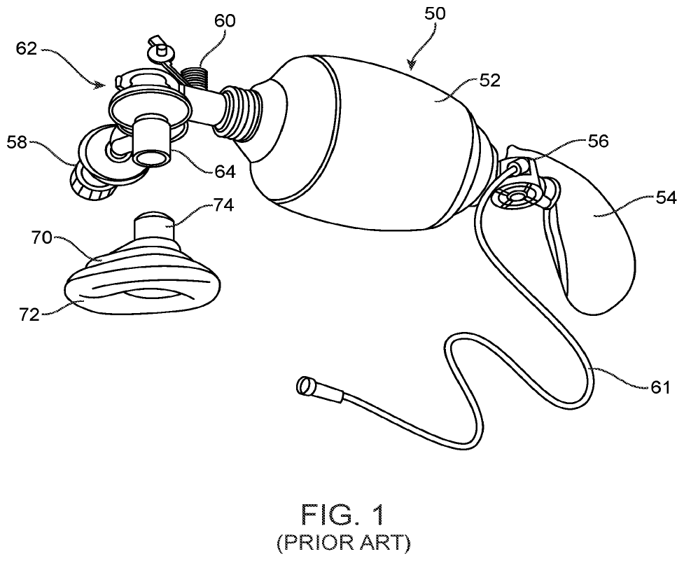

FIGS. 2A-1 to 2A-5 shows an add-on sensor 202 (FIG. 2A-1) that can be detachably secured at existing BVM's inlet 206 (FIG. 2A-3). The inlet 206 is not regulated and can vary from manufacturer to manufacturer to optimize performance. In majority of the cases the inlet 206 uses three valves. It comprises of the reservoir connector 204 (FIGS. 2A-2, 2A-3, and FIG. 2A-4), oxygen tube connection 210 (FIGS. 2A-3 and FIG. 2A-4), suction valve 207 (FIGS. 2A-3 and FIG. 2A-4), to enable air entry into the inlet 204, overpressure valve 209 (FIGS. 2A-3 and FIG. 2A-4) to enable pressure release by permitting air to be release to the atmosphere. The sensor 202 at the inlet 206 can measure pressure, flow, volume, time, temperature. The sensor 202 can off-the-shelf or custom made. Unlike bag connector port 64 on the manifold and connector fitting 74 on breathing mask in FIG. 1, reservoir connector 204 is not standardized. Bag manufactures use ports with inner diameter between 22 mm to 30 mm ID. As shown in FIG. 2C, an off-the-shelf sensor 222 with mating connectors of optimal diameter can be used at the reservoir connector 204. Experimentally, during standard resuscitator operation while manual ventilating only with ambient air, maximum volume of air enters the inlet 206 via reservoir connector 204 due to larger diameters. Thus, an off-the-shelf sensor 202/222 is acceptable functionally to solve the problem. The primary component of the inlet 206 enabling our application is the one-way valve 208 (FIG. 2A-5). The one-way valve 208 prevents air from leaving the bag through the inlet 206 during compression. Thus, the airflow parameters like tidal volume, rate, and minute ventilation can be measured by documenting the air that enters the bag through the inlet 206.

FIG. 2B shows the application of the invention of utilizing sensor at the inlet 206 of the bag. These sensors enable a) reusability b) minimization of deadspace c) reduction of torque on the airway. Oxygenated or Uncontaminated air 216 enters the bag and reaches the patient. When the patient exhales Contaminated air 218, the air does not enter the manifold 62 in FIG. 1 due a one-way valve at patient port 214. It creates a distinct Contaminated region 2020 by separating Deoxygenated or Contaminated air from the Oxygenated or Uncontaminated air. All commercially available airflow sensors lie in the Contaminated region 2020. Increase in the Contaminated region 2020 corresponds to higher dead space and a larger torque. The invention minimizes the Contaminated region 2020 while offering measurement functionality and reusability. A filter 212 can be added at the junction of sensor 212 and the inlet 206 as an added safety net for minimizing contaminated region.

FIG. 2C shows an implementation of an off-the-shelf flow sensor 222/202 that can be used to detect tidal volume, rate, and minute ventilation. The sensor 222 can have capabilities that turn it into a platform that is capable of storing, displaying, and transmitting airflow information with a wire or wirelessly. The platform 224 can alert the user or provide feedback to change technique. In another implementation sensor 224 can be integrated with sensors at exhalation port at the site of PEEP valve 58 or at manifold 62 to document mask leak or air entering the patient.

FIG. 2D shows another implementation of an off-the shelf sensor 222 with a pressure measurement port 226. The pressure measurement port 226 connects directly to the bag chamber 52 that communicates with the patient's lungs or to document chamber pressure at the sensor. Documentation of pressures in the bag chamber 52 helps quantify lung compliance and resistance, and when paired with readings from sensor 222, helps predict predicting tidal volume, i.e., air entering the lung based or new mask leaks.

FIG. 3. Shows a modified BVM with a modified inlet 302 optimized for our application with the three-valve system reservoir for normal BVM operation. It further comprises of a detachable/reusable flow sensor platform 306 with or without a display and an internally communicating pressure probe 304.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure. Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

1. An assembly for conveying breathing gas to a subject, comprising:
a bag configured to be attached to deliver a breathing gas to a manifold and having an air inlet and an air outlet, wherein then air inlet includes a one-way valve that allows air to flow into the bag from the ambient as the bag expands;
the manifold connected to the air outlet of the bag;
a breathing interface configured to be attached to an air outlet of the manifold to receive the breathing gas from the manifold; and
a sensor configured to be secured to the air inlet on the bag such that the sensor can sense air entering the bag while the sensor remains isolated from exhaled air.

2. The assembly of claim 1, wherein the sensor is configured to measure any one of pressure, volumetric flow rate, mass flow rate, and temperature.

3. The assembly of claim 1, wherein the sensor is configured to be sterilized by any one of heat, chemical, or gas sterilization.

4. The assembly of claim 1, wherein the sensor is configured to be detachably secured to the air inlet on the bag.

5. The assembly of claim 1, wherein the sensor is configured to be non-detachably secured to the air inlet on the bag.

6. The assembly of claim 1, wherein the sensor is configured to store or transmit information with a cable or wirelessly.

7. The assembly of claim 1, wherein the sensor is configured to alert the user or provide feedback based on the readings.

8. The assembly of claim 1, wherein the sensor is configured to calculate at least one of tidal volume, rate, or minute ventilation.

9. The assembly of claim 1, further comprising a bag pressure sensor, wherein the bag pressure sensor is configured calculate lung compliance and resistance.

10. A method of providing a breathing gas to a subject comprising:
delivering breathing gas to the subject via a first bag and a subject interface, the first bag having an inlet and an outlet in communication with the subject interface,
detachably securing at least one sensor to the inlet,
sensing, via the at least one sensor, air entering the first bag while remaining isolated from exhaled air.

11. The method of claim 10, further comprising disposing the first bag.

12. The method of claim 10, further comprising reusing the at least one sensor with a second bag.

13. The method of claim 10, further comprising measuring any one or more of pressure, volumetric flow rate, mass flow rate, or temperature using the flow sensor.

14. The method of claim 10, further comprising sterilizing the flow sensor by any one or more of heat, chemical, or gas sterilization.

15. The method of claim 10, wherein the flow sensor alerts the user, provides feedback based on the readings, or both.

16. The method of claim 10, further comprising calculating at least one or more of tidal volume, rate, or minute ventilation.

17. The method of claim 10, wherein the first bag comprises a bag pressure sensor.

18. The method of claim 10, further comprising calculating lung compliance and resistance.

19. The method of claim 10, wherein delivering breathing gas further comprises limiting a gas flow via a flow-limiting valve.

20. The method of claim 10, wherein the breathing gas is delivered via the outlet upon compression of the first bag and the first bag is refilled with breathing gas via the inlet upon release of the compression.
